SEQUENCE LISTING (97 sequences)

SEQ ID NO: 1 - DNA, 12 bp, synthetic construct
  source 1..12: mol_type = other DNA
cttgggtctcgt

SEQ ID NO: 2 - DNA, 12 bp, synthetic construct
  source 1..12: mol_type = other DNA
cgtgccttgttt

SEQ ID NO: 3 - DNA, 12 bp, synthetic construct
  source 1..12: mol_type = other DNA
tttgcgggttct

SEQ ID NO: 4 - DNA, 12 bp, synthetic construct
  source 1..12: mol_type = other DNA
tctggtccctct

SEQ ID NO: 5 - DNA, 26 bp, synthetic construct
  source 1..26: mol_type = other DNA
cacagaagccgacaacgagacccaag

SEQ ID NO: 6 - DNA, 26 bp, synthetic construct
  source 1..26: mol_type = other DNA
agcagcaagaaccaaaacaaggcacg

SEQ ID NO: 7 - DNA, 26 bp, synthetic construct
  source 1..26: mol_type = other DNA
ggagaaccgaaccaagaacccgcaaa

SEQ ID NO: 8 - DNA, 26 bp, synthetic construct
  source 1..26: mol_type = other DNA
acgcgaaaccaccaagagggaccaga

SEQ ID NO: 9 - DNA, 40 bp, synthetic construct
  source 1..40: mol_type = other DNA
agcgcgaagaaacaacgagacccaagcaaaacaaggcacg

SEQ ID NO: 10 - DNA, 40 bp, synthetic construct
  source 1..40: mol_type = other DNA
aagcacgagaagcaacgagacccaagcaagaacccgcaaa

SEQ ID NO: 11 - DNA, 40 bp, synthetic construct
  source 1..40: mol_type = other DNA
aggccacaaccacaacgagacccaagcaagagggaccaga

SEQ ID NO: 12 - DNA, 40 bp, synthetic construct
  source 1..40: mol_type = other DNA
gcacgccaacaacaaaacaaggcacgcaagaacccgcaaa

SEQ ID NO: 13 - DNA, 40 bp, synthetic construct
  source 1..40: mol_type = other DNA
cacagagcgagacaaaacaaggcacgcaagagggaccaga

SEQ ID NO: 14 - DNA, 40 bp, synthetic construct
  source 1..40: mol_type = other DNA
gagcaaacgacccaagaacccgcaaacaagagggaccaga

SEQ ID NO: 15 - DNA, 54 bp, synthetic construct
  source 1..54: mol_type = other DNA
acgcacgcaagacaacgagacccaagcaaaacaaggcacgcaagaacccgcaaa

SEQ ID NO: 16 - DNA, 54 bp, synthetic construct
  source 1..54: mol_type = other DNA
agcgaccgacaacaacgagacccaagcaaaacaaggcacgcaagagggaccaga

SEQ ID NO: 17 - DNA, 54 bp, synthetic construct
  source 1..54: mol_type = other DNA
aacagaaccacccaacgagacccaagcaagaacccgcaaacaagagggaccaga

SEQ ID NO: 18 - DNA, 54 bp, synthetic construct
  source 1..54: mol_type = other DNA
aaacaggcagaccaaaacaaggcacgcaagaacccgcaaacaagagggaccaga

SEQ ID NO: 19 - DNA, 68 bp, synthetic construct
  source 1..68: mol_type = other DNA
ccagcaaacagacaacgagacccaagcaaaacaaggcacgcaagaacccgcaaacaagagggaccaga

SEQ ID NO: 20 - DNA, 26 bp, synthetic construct
  source 1..26: mol_type = other DNA
ccaagcagcgaacaacgagacccaag

SEQ ID NO: 21 - DNA, 26 bp, synthetic construct
  source 1..26: mol_type = other DNA
gaaccggacacacaaaacaaggcacg

SEQ ID NO: 22 - DNA, 26 bp, synthetic construct
  source 1..26: mol_type = other DNA
aggcaccaacaccaagaacccgcaaa

SEQ ID NO: 23 - DNA, 26 bp, synthetic construct
  source 1..26: mol_type = other DNA
aacggcaaaggacaagagggaccaga

SEQ ID NO: 24 - DNA, 40 bp, synthetic construct
  source 1..40: mol_type = other DNA
aagcaggaccgacaacgagacccaagcaaaacaaggcacg

SEQ ID NO: 25 - DNA, 40 bp, synthetic construct
  source 1..40: mol_type = other DNA
cagagaaggcgacaacgagacccaagcaagaacccgcaaa

SEQ ID NO: 26 - DNA, 40 bp, synthetic construct
  source 1..40: mol_type = other DNA
aacagcaggaggcaacgagacccaagcaagagggaccaga

SEQ ID NO: 27 - DNA, 40 bp, synthetic construct
  source 1..40: mol_type = other DNA
agaagcagacgacaaaacaaggcacgcaagaacccgcaaa

SEQ ID NO: 28 - DNA, 40 bp, synthetic construct
  source 1..40: mol_type = other DNA
acgcacaaggaacaaaacaaggcacgcaagagggaccaga

SEQ ID NO: 29 - DNA, 40 bp, synthetic construct
  source 1..40: mol_type = other DNA
aggaagacggagcaagaacccgcaaacaagagggaccaga

SEQ ID NO: 30 - DNA, 54 bp, synthetic construct
  source 1..54: mol_type = other DNA
caagaacgcgcacaacgagacccaagcaaaacaaggcacgcaagaacccgcaaa

SEQ ID NO: 31 - DNA, 54 bp, synthetic construct
  source 1..54: mol_type = other DNA
aaagcggaaggccaacgagacccaagcaaaacaaggcacgcaagagggaccaga

SEQ ID NO: 32 - DNA, 54 bp, synthetic construct
  source 1..54: mol_type = other DNA
cgaacagacggacaacgagacccaagcaagaacccgcaaacaagagggaccaga

SEQ ID NO: 33 - DNA, 54 bp, synthetic construct
  source 1..54: mol_type = other DNA
aaggagacgagccaaaacaaggcacgcaagaacccgcaaacaagagggaccaga

SEQ ID NO: 34 - DNA, 68 bp, synthetic construct
  source 1..68: mol_type = other DNA
gacagaacgcaccaacgagacccaagcaaaacaaggcacgcaagaacccgcaaacaagagggaccaga

SEQ ID NO: 35 - DNA, 26 bp, synthetic construct
  source 1..26: mol_type = other DNA
aaaggacggcagcaacgagacccaag

SEQ ID NO: 36 - DNA, 26 bp, synthetic construct
  source 1..26: mol_type = other DNA
agaccgcacacacaaaacaaggcacg

SEQ ID NO: 37 - DNA, 26 bp, synthetic construct
  source 1..26: mol_type = other DNA
agacgacaggaacaagaacccgcaaa

SEQ ID NO: 38 - DNA, 26 bp, synthetic construct
  source 1..26: mol_type = other DNA
accagcggaaagcaagagggaccaga

SEQ ID NO: 39 - DNA, 40 bp, synthetic construct
  source 1..40: mol_type = other DNA
ggcagccacaaacaacgagacccaagcaaaacaaggcacg

SEQ ID NO: 40 - DNA, 40 bp, synthetic construct
  source 1..40: mol_type = other DNA
gaccaacaaacgcaacgagacccaagcaagaacccgcaaa

SEQ ID NO: 41 - DNA, 40 bp, synthetic construct
  source 1..40: mol_type = other DNA
cgaacacagacgcaacgagacccaagcaagagggaccaga

SEQ ID NO: 42 - DNA, 40 bp, synthetic construct
  source 1..40: mol_type = other DNA
caaggcaagcaacaaaacaaggcacgcaagaacccgcaaa

SEQ ID NO: 43 - DNA, 40 bp, synthetic construct
  source 1..40: mol_type = other DNA
acaaacggccagcaaaacaaggcacgcaagagggaccaga

SEQ ID NO: 44 - DNA, 40 bp, synthetic construct
  source 1..40: mol_type = other DNA
cgaagcacacagcaagaacccgcaaacaagagggaccaga

SEQ ID NO: 45 - DNA, 54 bp, synthetic construct
  source 1..54: mol_type = other DNA
agacagaaacggcaacgagacccaagcaaaacaaggcacgcaagaacccgcaaa

SEQ ID NO: 46 - DNA, 54 bp, synthetic construct
  source 1..54: mol_type = other DNA
aagaggcagagccaacgagacccaagcaaaacaaggcacgcaagagggaccaga

SEQ ID NO: 47 - DNA, 54 bp, synthetic construct
  source 1..54: mol_type = other DNA
accaccagagcacaacgagacccaagcaagaacccgcaaacaagagggaccaga

SEQ ID NO: 48 - DNA, 54 bp, synthetic construct
  source 1..54: mol_type = other DNA
acaaggcgagaccaaaacaaggcacgcaagaacccgcaaacaagagggaccaga

SEQ ID NO: 49 - DNA, 68 bp, synthetic construct
  source 1..68: mol_type = other DNA
aagacaacagcgcaacgagacccaagcaaaacaaggcacgcaagaacccgcaaacaagagggaccaga

SEQ ID NO: 50 - DNA, 72 bp, synthetic construct
  source 1..72: mol_type = other DNA
  modified_base 1: mod_base = OTHER; note = 5' phosphorylated nucleotide
tataccgcgccaacacccgttggttggttcttccgggtctctgtgccgtcctgcttggctaatcgctgagga

SEQ ID NO: 51 - DNA, 72 bp, synthetic construct
  source 1..72: mol_type = other DNA
  modified_base 1: mod_base = OTHER; note = 5' phosphorylated nucleotide
ttgggataagggctgtgttgttcttcgtggtcgtgcgtcgtcgggccgtcctgcttacaagtctgccgacca

SEQ ID NO: 52 - DNA, 72 bp, synthetic construct
  source 1..72: mol_type = other DNA
  modified_base 1: mod_base = OTHER; note = 5' phosphorylated nucleotide
tataggcaggttgggaggccgtggtttcgggtgtgttcttctgggccgtcctgcttagaagtacgcgtgcct

SEQ ID NO: 53 - DNA, 72 bp, synthetic construct
  source 1..72: mol_type = other DNA
  modified_base 1: mod_base = OTHER; note = 5' phosphorylated nucleotide
ttacggacgcaagggattctctcttgttgggcttgtgtggctcggccgtcctgcttaaggcggagagtacga

SEQ ID NO: 54 - DNA, 72 bp, synthetic construct
  source 1..72: mol_type = other DNA
  modified_base 1: mod_base = OTHER; note = 5' phosphorylated nucleotide
tagcgaagaaagggctttccctcgcttgcgtctctttcgttgctgccgtcctgcttagccctcttgagacca

SEQ ID NO: 55 - DNA, 72 bp, synthetic construct
  source 1..72: mol_type = other DNA
  modified_base 1: mod_base = OTHER; note = 5' phosphorylated nucleotide
aacccgaaagctccagttgcttgtttctcgggttggtgtgcgctgccgtcctgcttaccgtatggccagaca

SEQ ID NO: 56 - DNA, 72 bp, synthetic construct
  source 1..72: mol_type = other DNA
  modified_base 1: mod_base = OTHER; note = 5' phosphorylated nucleotide
aatgagcctctgacggggttggcctcttccgtttgtgtggctctgccgtcctgcttacctaaccgcccaaga

SEQ ID NO: 57 - DNA, 72 bp, synthetic construct
  source 1..72: mol_type = other DNA
  modified_base 1: mod_base = OTHER; note = 5' phosphorylated nucleotide
aagccctgccataaacgttctctgtcgttcggcggttccttgcggccgtcctgcttcttggccaggagaaca

SEQ ID NO: 58 - DNA, 72 bp, synthetic construct
  source 1..72: mol_type = other DNA
  modified_base 1: mod_base = OTHER; note = 5' phosphorylated nucleotide
aaggaggtcggaccattcggtttctcttccggttgggttgcgcggccgtcctgcttttgagcgagaagggct

SEQ ID NO: 59 - DNA, 72 bp, synthetic construct
  source 1..72: mol_type = other DNA
  modified_base 1: mod_base = OTHER; note = 5' phosphorylated nucleotide
aagacatgggccaagggtccgccgtttggtcttggtgttgtcctgccgtcctgcttagtgtgggctaatgga

SEQ ID NO: 60 - DNA, 72 bp, synthetic construct
  source 1..72: mol_type = other DNA
  modified_base 1: mod_base = OTHER; note = 5' phosphorylated nucleotide
ttgcgggctgactcatggttgcgccctgtctttcccgtctttctgccgtcctgcttaaccaggaatgggact

SEQ ID NO: 61 - DNA, 72 bp, synthetic construct
  source 1..72: mol_type = other DNA
  modified_base 1: mod_base = OTHER; note = 5' phosphorylated nucleotide
aagacggtaccgcatgtctgccttgtgttccttgcgttgggcctgccgtcctgcttcccaacaacagagcct

SEQ ID NO: 62 - DNA, 72 bp, synthetic construct
  source 1..72: mol_type = other DNA
  modified_base 1: mod_base = OTHER; note = 5' phosphorylated nucleotide
taggaccaccaatagctctgctggtgttgtccgctgtttggtccgccgtcctgcttacaggatggcacgtgt

SEQ ID NO: 63 - DNA, 72 bp, synthetic construct
  source 1..72: mol_type = other DNA
  modified_base 1: mod_base = OTHER; note = 5' phosphorylated nucleotide
taacaagggagtggcatccggttcgttcttgtccttgtcgcctggccgtcctgcttatgtaactggcgcgct

SEQ ID NO: 64 - DNA, 72 bp, synthetic construct
  source 1..72: mol_type = other DNA
  modified_base 1: mod_base = OTHER; note = 5' phosphorylated nucleotide
atgcacaatggcgactttcttccttcgtgctgctccgtgttcgcgccgtcctgcttcagggtccaggaagaa

SEQ ID NO: 65 - DNA, 72 bp, synthetic construct
  source 1..72: mol_type = other DNA
  modified_base 1: mod_base = OTHER; note = 5' phosphorylated nucleotide
tagaaggagtgccaacgggtgctcgcttcggtcgtgttcttgtggccgtcctgcttacgagagaccacccat

SEQ ID NO: 66 - DNA, 72 bp, synthetic construct
  source 1..72: mol_type = other DNA
  modified_base 1: mod_base = OTHER; note = 5' phosphorylated nucleotide
ttagtgcgcctgggatgctccctctggtttcttggtcgtggcttgccgtcctgctttcaccgggaatagcca

SEQ ID NO: 67 - DNA, 72 bp, synthetic construct
  source 1..72: mol_type = other DNA
  modified_base 1: mod_base = OTHER; note = 5' phosphorylated nucleotide
aatacaacggcctgccttgctccttcctgttgtgttgcctgtcggccgtcctgcttctgagacggaactcca

SEQ ID NO: 68 - DNA, 72 bp, synthetic construct
  source 1..72: mol_type = other DNA
  modified_base 1: mod_base = OTHER; note = 5' phosphorylated nucleotide
ttgaagctcctggccagcgctttggtctccctgggtttcctggtgccgtcctgctttcaggaacgggtacaa

SEQ ID NO: 69 - DNA, 72 bp, synthetic construct
  source 1..72: mol_type = other DNA
  modified_base 1: mod_base = OTHER; note = 5' phosphorylated nucleotide
taagaggagccgtaactgttgttctgccgcctcttctcgtcgtggccgtcctgcttggacacgaagggctta

SEQ ID NO: 70 - DNA, 72 bp, synthetic construct
  source 1..72: mol_type = other DNA
  modified_base 1: mod_base = OTHER; note = 5' phosphorylated nucleotide
taggacgatgcgacagggctcgtgtttctgcgcgtcgtttgtccgccgtcctgcttgccggcagatgaagaa

SEQ ID NO: 71 - DNA, 72 bp, synthetic construct
  source 1..72: mol_type = other DNA
  modified_base 1: mod_base = OTHER; note = 5' phosphorylated nucleotide
tacgagtgggaagacatgtggcctcgtttggtcttcgcgtttgtgccgtcctgctttgcaaagccgggcata

SEQ ID NO: 72 - DNA, 72 bp, synthetic construct
  source 1..72: mol_type = other DNA
  modified_base 1: mod_base = OTHER; note = 5' phosphorylated nucleotide
atagggccgcgagaatttcttgtctgcccgttgcgctttctcttgccgtcctgcttaggatctgcaggagta

SEQ ID NO: 73 - DNA, 72 bp, synthetic construct
  source 1..72: mol_type = other DNA
  modified_base 1: mod_base = OTHER; note = 5' phosphorylated nucleotide
atctacactagccgccgttctcgttcttccttccttgcgcggttgccgtcctgctttaggcaatgcagcgga

SEQ ID NO: 74 - DNA, 40 bp, synthetic construct
  source 1..40: mol_type = other DNA
cgttggttggttcttccgggtctctgaccttgggtctcgt

SEQ ID NO: 75 - DNA, 40 bp, synthetic construct
  source 1..40: mol_type = other DNA
ttgttcttcgtggtcgtgcgtcgtcgaccgtgccttgttt

SEQ ID NO: 76 - DNA, 40 bp, synthetic construct
  source 1..40: mol_type = other DNA
gccgtggtttcgggtgtgttcttctgactttgcgggttct

SEQ ID NO: 77 - DNA, 40 bp, synthetic construct
  source 1..40: mol_type = other DNA
tctctcttgttgggcttgtgtggctcactctggtccctct

SEQ ID NO: 78 - DNA, 54 bp, synthetic construct
  source 1..54: mol_type = other DNA
tccctcgcttgcgtctctttcgttgcaccttgggtctcgtaccgtgccttgttt

SEQ ID NO: 79 - DNA, 54 bp, synthetic construct
  source 1..54: mol_type = other DNA
tgcttgtttctcgggttggtgtgcgcaccttgggtctcgtactttgcgggttct

SEQ ID NO: 80 - DNA, 54 bp, synthetic construct
  source 1..54: mol_type = other DNA
gttggcctcttccgtttgtgtggctcaccttgggtctcgtactctggtccctct

SEQ ID NO: 81 - DNA, 54 bp, synthetic construct
  source 1..54: mol_type = other DNA
ttctctgtcgttcggcggttccttgcaccgtgccttgtttactttgcgggttct

SEQ ID NO: 82 - DNA, 54 bp, synthetic construct
  source 1..54: mol_type = other DNA
cggtttctcttccggttgggttgcgcaccgtgccttgtttactctggtccctct

SEQ ID NO: 83 - DNA, 54 bp, synthetic construct
  source 1..54: mol_type = other DNA
tccgccgtttggtcttggtgttgtccactttgcgggttctactctggtccctct

SEQ ID NO: 84 - DNA, 68 bp, synthetic construct
  source 1..68: mol_type = other DNA
gttgcgccctgtctttcccgtctttcaccttgggtctcgtaccgtgccttgtttactttgcgggttct

SEQ ID NO: 85 - DNA, 68 bp, synthetic construct
  source 1..68: mol_type = other DNA
ctgccttgtgttccttgcgttgggccaccttgggtctcgtaccgtgccttgtttactctggtccctct

SEQ ID NO: 86 - DNA, 68 bp, synthetic construct
  source 1..68: mol_type = other DNA
ctgctggtgttgtccgctgtttggtcaccttgggtctcgtactttgcgggttctactctggtccctct

SEQ ID NO: 87 - DNA, 68 bp, synthetic construct
  source 1..68: mol_type = other DNA
ccggttcgttcttgtccttgtcgcctaccgtgccttgtttactttgcgggttctactctggtccctct

SEQ ID NO: 88 - DNA, 82 bp, synthetic construct
  source 1..82: mol_type = other DNA
tcttccttcgtgctgctccgtgttcgaccttgggtctcgtaccgtgccttgtttactttgcgggttctactctggtccctct

SEQ ID NO: 89 - DNA, 40 bp, synthetic construct
  source 1..40: mol_type = other DNA
ggtgctcgcttcggtcgtgttcttgtaccttgggtctcgt

SEQ ID NO: 90 - DNA, 40 bp, synthetic construct
  source 1..40: mol_type = other DNA
ctccctctggtttcttggtcgtggctaccgtgccttgttt

SEQ ID NO: 91 - DNA, 40 bp, synthetic construct
  source 1..40: mol_type = other DNA
tgctccttcctgttgtgttgcctgtcactttgcgggttct

SEQ ID NO: 92 - DNA, 40 bp, synthetic construct
  source 1..40: mol_type = other DNA
cgctttggtctccctgggtttcctggactctggtccctct

SEQ ID NO: 93 - DNA, 54 bp, synthetic construct
  source 1..54: mol_type = other DNA
gttgttctgccgcctcttctcgtcgtaccttgggtctcgtaccgtgccttgttt

SEQ ID NO: 94 - DNA, 54 bp, synthetic construct
  source 1..54: mol_type = other DNA
gctcgtgtttctgcgcgtcgtttgtcaccttgggtctcgtactttgcgggttct

SEQ ID NO: 95 - DNA, 54 bp, synthetic construct
  source 1..54: mol_type = other DNA
gtggcctcgtttggtcttcgcgtttgaccttgggtctcgtactctggtccctct

SEQ ID NO: 96 - DNA, 54 bp, synthetic construct
  source 1..54: mol_type = other DNA
tcttgtctgcccgttgcgctttctctaccgtgccttgtttactttgcgggttct

SEQ ID NO: 97 - DNA, 54 bp, synthetic construct
  source 1..54: mol_type = other DNA
ttctcgttcttccttccttgcgcggtaccgtgccttgtttactctggtccctct

(19) United States
(12) Patent Application Publication
(10) Pub. No.: US 2026/0201456 A1
(43) Pub. Date: Jul. 16, 2026

(54) IN SITU DETECTION OF BARCODES

(71) Applicant: Genentech, Inc., South San Francisco, CA (US)

(72) Inventor: Eric Scott LUBECK, San Francisco, CA (US)

(73) Assignee: Genentech, Inc., South San Francisco, CA (US)

(21) Appl. No.: 19/427,837

(22) Filed: Dec. 19, 2025

Related U.S. Application Data
(63) Continuation of application No. PCT/US2024/034716, filed on Jun. 20, 2024.
(60) Provisional application No. 63/522,809, filed on Jun. 23, 2023.

(51) Int. Cl. C12Q 1/6841; C12N 15/10; C12N 15/86; C12Q 1/6844; C12Q 1/6874; G01N 1/30

(52) U.S. Cl. CPC C12Q 1/6841; C12N 15/1065; C12N 15/86; C12Q 1/6844; C12Q 1/6874; G01N 1/30; C12N 2740/15041

(57) ABSTRACT

The current disclosure provides a method suitable for detecting barcode sequences in a biological sample in situ. Advantageously, the method can be effectively used to detect barcode sequences in formalin-fixed paraffin-embedded (FFPE) tissue samples.

### CROSS-REFERENCE TO RELATED APPLICATIONS

This application is a continuation of International Application No. PCT/US2024/034716, filed Jun. 20, 2024, which claims benefit of priority to U.S. Patent Application No. 63/522,809, filed Jun. 23, 2023, each of which is incorporated herein by reference in its entirety.

### REFERENCE TO SEQUENCE LISTING

This application contains a Sequence Listing which has been submitted electronically in XML format and is hereby incorporated by reference in its entirety. Said XML copy, created on Dec. 15, 2025, is named P38032-US-1_SL.xml and is 102,481 bytes in size.

### INCORPORATION BY REFERENCE

All publications, patents, and patent applications cited herein are incorporated by reference to the same extent as if each individual publication, patent, or patent application was specifically and individually indicated to be incorporated by reference. In the event of a conflict between a term herein and a term in an incorporated reference, the term herein controls.

### BACKGROUND

Understanding of gene function has been improving given the recent technological advances coupling pooled genetic perturbations with in vivo screening methods. For example, in situ readout of a pooled screening has been reported in isolated cells (Feldman et al., 2019). This pooled screening method has not been shown to work in vivo. It requires pooled cloning and pooled transfection. The screening is achieved via direct guide sequencing. On the other hand, multiplexed epitope staining has been reported (Wroblewska et al. 2018 and Dhainaur et al., 2022). Although this method was reported to work in vivo, it requires pooled two-step cloning and arrayed transfection. Additionally, the generated epitopes could be immunogenic, limiting the methods' application.

However, there is still a need in the art for developing in vivo screening methods capable of more effectively capturing biological complexity in its native environment. Such methods can be used to, for example, facilitate drug discovery in the disease-relevant context. The present disclosure addresses these needs.

### SUMMARY

The current disclosure provides methods for determining barcode sequences in situ. For example, the described systems and methods are suitable for detecting barcode sequences in formalin-fixed paraffin-embedded (FFPE) tissue samples.

In one aspect, provided is a method for detecting barcode sequences in a biological sample in situ, comprising the steps of: (1) processing a biological sample comprising a plurality of cells, wherein each cell of the plurality of cells comprises an oligonucleotide having a single barcode sequence of 20 to 40 or 20 to 60 nucleotides, so that a plurality of barcode molecules, each of which comprises the barcode sequence, are amplified in the plurality of cells of the biological sample in situ, and (2) detecting the plurality of barcode molecules so that the barcode sequences are located in situ. Detecting the plurality of barcode molecules in situ can be achieved by in situ nucleic acid sequencing or fluorescence in situ hybridization.

The biological sample can be obtained from a mammal; and for example, the biological sample can be a mammalian tumor tissue. The method may further comprise a step of fixing the mammalian tumor tissue before the processing step. The fixed mammalian tumor tissue can be a formalin-fixed paraffin-embedded (FFPE) tissue.

In another aspect, the biological sample for barcode detection can be obtained by preparing a pooled viral vector library comprising the oligonucleotides; transforming the pooled viral vector library to a plurality of cells; and obtaining a biological sample comprising the plurality of transformed cells. The pooled viral vector library can be delivered to a mammal so that a plurality of mammalian cells are transformed with the pooled viral vector library. Alternatively, the pooled viral vector library can be transformed into a plurality of cultured mammalian cells, wherein the transformed cultured mammalian cells are transferred to a mammal. The pooled viral vector library can be constructed from a retrovirus vector, a lentivirus vector, a gammaretroviral vector, an adenovirus vector, or an adeno-associated virus (AAV) vector. The retrovirus vector, the lentivirus vector, or the gammaretroviral vector can be a non-integrating viral vector or a self-inactivating retroviral vector. The viral vector may further comprise a cDNA or a CRISPR gRNA.

The oligonucleotide, which contains the barcode sequence, may further comprise a CRISPR gRNA. Additionally, the barcode sequence can be operably linked to a promoter, wherein the prompter can be a bacteriophage T3 promoter, a bacteriophage T7 promoter, or a bacteriophage SP6 promoter. The barcode sequence may have a GC content of about 30% to about 70%; and may have a Tm in a range of about 42° C. to about 75° C., and typically about 60° C. or greater.

In yet another aspect, provided is a method for detecting barcode sequences further comprising the following step before the processing step: (a) transcribing the barcode sequence to produce an RNA molecule; (b) annealing the RNA molecule to a padlock probe, wherein the padlock probe comprises an imaging barcode flanked by two RNA-binding sequences, and wherein each RNA-binding sequence is complementary to a section of the barcode sequence; (c) ligating the padlock probe to form a single chain circular DNA molecule, and (d) performing rolling circle replication of the single chain circular DNA molecule to amplify the barcode molecule. The padlock probe may comprise a spacer oligonucleotide placed between the imaging barcode and at least one of the two RNA-binding sequences. The imaging barcode may have a length of 28 bases. Each of the two RNA-binding sequences may (1) have a length of 7-20 bases; (2) have a GC content of about 30% to about 70%; (3) contain only guanosine (G), cytosine (C), and adenine (A); and/or have a Tm in a range of about 42° C. to about 65° C. The padlock probe may be designed so that an interaction between the padlock probe and the barcode sequence has a Levenshtein distance greater than 4 and a Tm greater than 42° C.

In a further aspect, detecting the plurality of barcode molecules may be achieved by performing (i) in situ sequencing of the barcode molecule, or (ii) in situ hybridization against the barcode molecule, a branched DNA bound to the barcode molecule, or the imaging barcode. The in situ hybridization can be fluorescence in situ hybridization (FISH).

### BRIEF DESCRIPTION OF THE DRAWINGS

FIG. 1A depicts a partial map of an exemplary construct that can be used for in vivo barcode detection. FIG. 1B depicts an exemplary scheme of barcode detection.

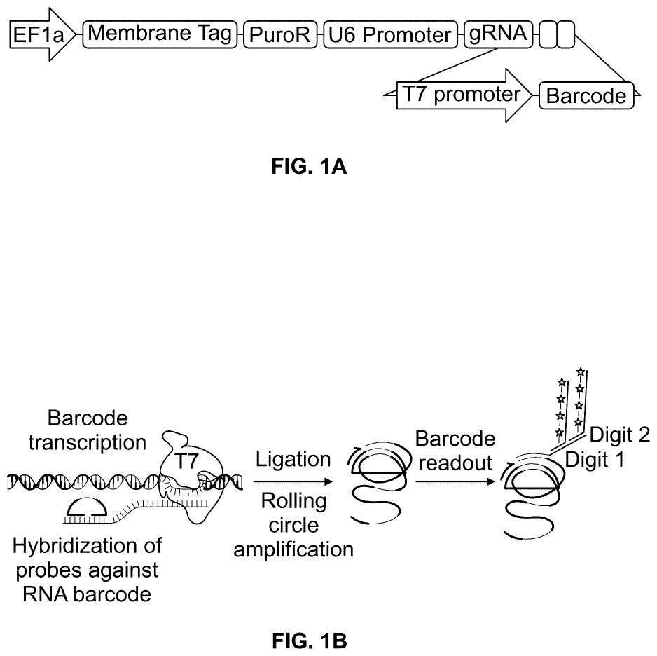

FIG. 2. depicts the validation of orthogonal barcode detection. DLD-1 cells were transduced with barcodes, 1 per well except for Lenti 3+4, and stained with probes for all barcodes with one probe for well. The results show that only the cells transduced with the specific barcode are stained by the appropriate probe.

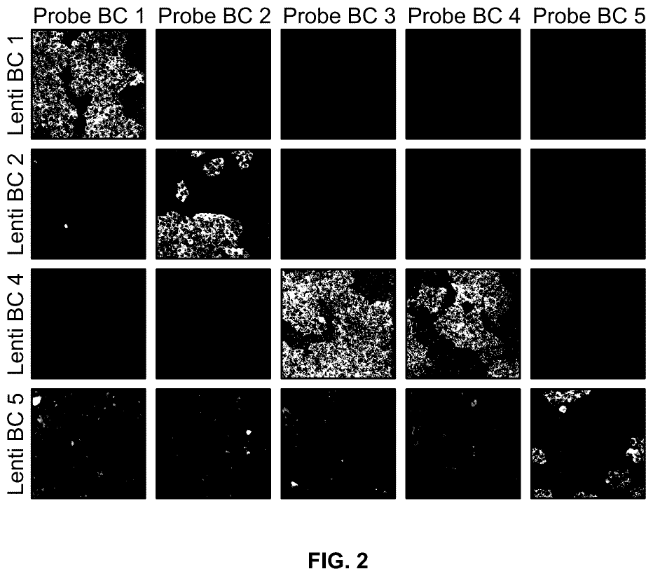

FIG. 3 depicts that transcription directed by the T7 RNA polymerase enables barcode detection when mRNA is degraded. Cells were crosslinked with formaldehyde for 15 minutes before the zombie protocol was developed for silencing-resistant OPS. Most cells develop major barcode nuclear foci. Barcodes diffuse between cells as expected but do not swamp out the nuclear foci encoding cell ID.

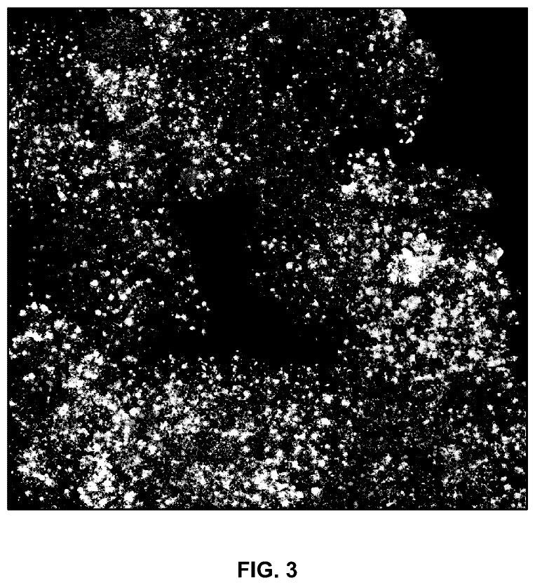

FIG. 4 depicts cryo-frozen colorectal cancer (CRC) organoids transduced and stained for the barcoding system. Organoids are grown in vivo after pooled lentiviral transduction, then fixed and stained to visualize barcode expression.

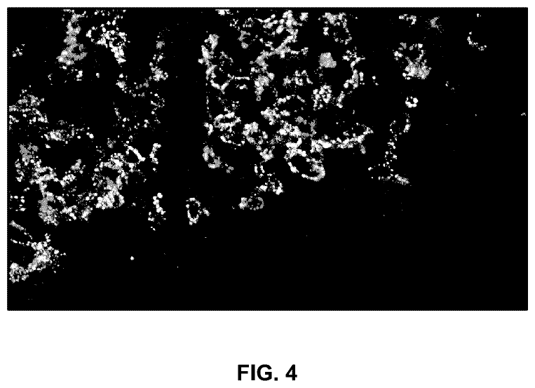

FIG. 5A depicts the experimental setup for Example 1. FIG. 5B depicts xenograft DLD-1 tumor growing in nude mice expressing 24 unique barcodes. Tissue was cryopreserved post-harvest, cut into 10 μm sections, and ultimately stained with the oligonucleotide barcodes as described in Example 1. Tissue was then imaged on a microscope according to the below protocol to generate the image. Each shade corresponds to a barcode that is genetically linked to a CRISPR sgRNA. The scalability of the method suggests that it is possible to simultaneously track more than 24 barcodes.

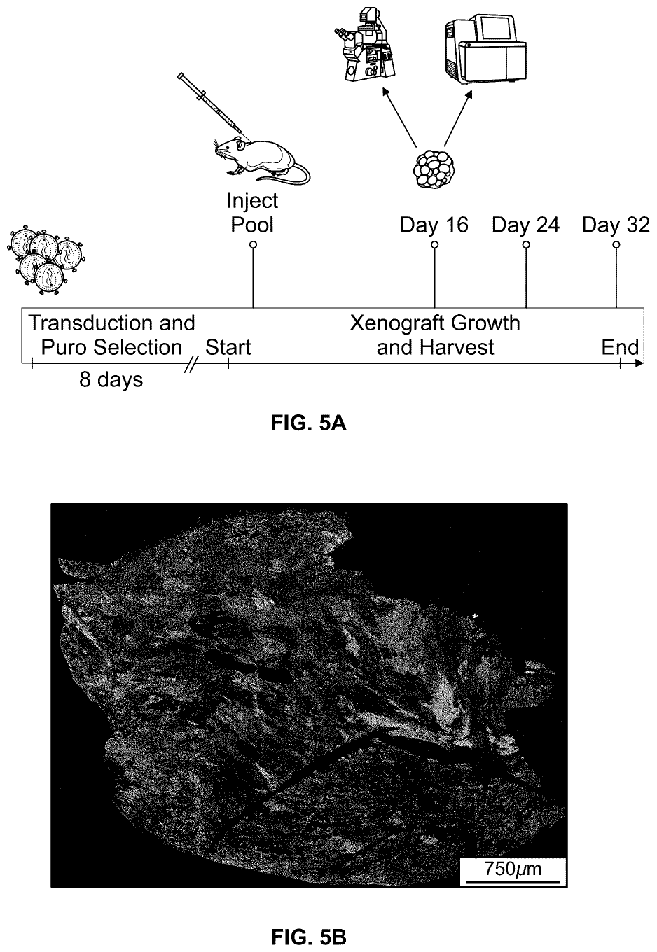

### DETAILED DESCRIPTION

### Definitions

Unless defined otherwise, technical and scientific terms used herein have the same meaning as commonly understood by one of ordinary skill in the art to which the present disclosure belongs. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Spring Harbor Press (Cold Spring Harbor, N.Y. 1989). For purposes of the present disclosure, the following terms are defined below.

As used in the specification, embodiments, and claims, the singular forms “a,” “an” and “the” include plural references unless the context clearly dictates otherwise. For example, the term “a cell” includes a plurality of cells, including mixtures thereof. Similarly, the use of “a compound” in a method as described herein contemplates using one or more compounds of this invention for such method unless the context clearly dictates otherwise.

As used herein, the term “comprising” is intended to mean that the compositions and methods include the recited elements, but not excluding others. “Consisting essentially of” when used herein to define compositions and methods, shall mean excluding other elements of any essential significance to the combination. Thus, a method consisting essentially of the recited steps as defined herein would not exclude additional steps that do not alter the method from yielding the claimed result. “Consisting of” shall mean in the context of a method excluding additional steps and limiting the claim only to the recited steps. Embodiments defined by each of these transition terms are within the scope of this invention.

Polypeptides and nucleic acids have a form of directionality when discussed in certain orientations. A nucleic acid is discussed in a 5′ to 3′ direction, or sense direction, which relates for example, to when a nucleic acid is translated into a polypeptide. A polypeptide is described in an amino-terminal (N-terminal or N-terminus) to carboxy-terminal (C-terminal or C-terminus) orientation.

The term “about” in relation to a reference numerical value, and its grammatical equivalents as used herein, can include the reference numerical value itself and a range of values plus or minus 10% from that reference numerical value. For example, the term “about 10” includes 10 and any amount from and including 9 to 11. In some cases, the term “about” in relation to a reference numerical value can also include a range of values plus or minus 10%, 9%, 8%, 7%, 6%, 5%, 4%, 3%, 2%, or 1% from that reference numerical value. In some embodiments, “about” in connection with a number or range measured by a particular method indicates that the given numerical value includes values determined by the variability of that method.

### Barcode, Barcode Expression, and Barcode Amplification

The single barcode sequence present in each of the plurality of cells can contain 20 to 60 nucleotides, for example, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides. Each barcode sequence typically may have a GC content in the range of about 30-70%, for example, a GC content of about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, and about 70%. Each barcode sequence typically may have a Tm in the range of about 37° C. to about 75° C., for example, a Tm of about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.

Each barcode sequence may be initially synthesized as an oligonucleotide of about 100 nucleotides, about 120 nucleotides, about 140 nucleotides, about 160 nucleotides, about 180 nucleotides, about 200 nucleotides, about 220 nucleotides, about 240 nucleotides, about 260 nucleotides, about 280 nucleotides, or about 300 nucleotides. The synthesized oligonucleotide may contain another functional fragment, e.g., a CRISPR gRNA or a promoter. The promoter can be a bacteriophage promoter, for example, a T3 promoter, a T7 promoter, or an SP6 promoter.

In some embodiments, the plurality of live cells may not express the barcode molecules (e.g., the barcode sequence is not transcribed in the live cell). The plurality of barcode molecules can be generated by: transcribing the barcode sequence in each of the plurality of fixed cells to generate the plurality of barcode molecules comprising the barcode sequence of the fixed cells. Transcribing the barcode sequences can be achieved by using a phage RNA polymerase when an appropriate promoter is placed upstream of the barcode sequence. The phage RNA polymerase can comprise a bacteriophage T3 RNA polymerase, a bacteriophage T7 RNA polymerase, a bacteriophage SP6 RNA polymerase, or a combination thereof.

The plurality of transcribed barcode sequences can be amplified to form a plurality of barcode molecules for detection. The number of barcode molecules amplified in each cell can be different in different implementations. The number of barcode molecules generated in each cell can be, can be about, can be at least, or can be at most, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or any number or any range between any two of these values.

The amplification of transcribed barcode sequences can be achieved with well-established methods, for example, using rolling circle replication with a padlock probe. For this, a padlock probe is provided to anneal to the transcribed barcode RNA molecule, wherein the padlock probe comprises two RNA-binding sequences, one located at the 5′ end and another located at the 3′ end, each of which is complementary to a section of the barcode sequence. The padlock probe is then ligated to form a single-chain circular DNA molecule, which is subsequently subject to the rolling circle replication to form single-chain circular DNA molecules, thereby achieving the amplification of the barcode molecules.

For the padlock probe, each of its two RNA-binding sequences typically may have a length of 7 bases, 8 bases, 9 bases, 10 bases, 11 bases, 12 bases, 13 bases, 14 bases, 15 bases, 16 bases, 17 bases, 18 bases, 19 bases, or 20 bases. Additionally, each of the two RNA-binding sequences may have a GC content in the range of about 30-70%, for example, a GC content of about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, and about 70%. Each RNA-binding sequence of a padlock probe may contain only guanosine (G), cytosine (C), and adenine (A). Additionally, each RNA-binding sequence of a padlock probe may have a Tm in the range of about 42° C. to about 65° C., for example, a Tm of about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., or about 65° C.

The padlock probe and the barcode sequence may be designed so that an interaction between the padlock probe and the barcode sequence has a Levenshtein distance greater than 2, for example, greater than 3, greater than 4, greater than 5, greater than 6, greater than 7, or greater than 8. The interaction between the padlock probe and the barcode sequence also may have a Tm greater than about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.

### Barcode Detection

The amplified barcode molecules can be detected in situ, for example, by in situ nucleic acid sequencing or fluorescence in situ hybridization.

In situ sequencing may be performed, for example, using the Spatially-resolved Transcript Amplicon Readout Mapping (STARmap) technique or any of its modified versions, with necessary adaptations. The original STARmap technique has been described in WO2019/199579 A1 and Wang et al., 2018. Modified versions of STARmap may also be used for in situ sequencing, such as described in WO2021/076770 A1 and WO2022/246269 A1. In general, STARmap methods and variations thereof utilize image-based in situ nucleic acid (DNA and/or RNA) sequencing technology using a sequencing-by-ligation process, specific signal amplification, hydrogel-tissue chemistry to turn biological tissue into a transparent sequencing chip, and associated data analysis pipelines to spatially-resolve highly-multiplexed gene detection at a subcellular and cellular level. In some embodiments, the methods described herein include spatial sequencing (e.g. reagents, chips or services) for biomedical research and clinical diagnostics with single-cell sensitivity.

In some embodiments, fluorescence in situ hybridization can be used to detect the amplified barcode molecules. The fluorescence in situ hybridization can be against the barcode molecule, a branched DNA bound to the barcode molecule, or an imaging barcode sequence present in the padlock probe used for amplification. For fluorescence in situ hybridization, the cells containing the amplified barcode molecules may be contacted with a plurality of detection probes, each of which comprises a barcode binding sequence and a fluorophore. The barcode binding sequence is a reverse complement (antisense) to a barcode sequence (or a portion thereof), the branched DNA bound to the barcode molecule, or the imaging barcode (or a portion thereof) so that the detection probe is capable of hybridizing with the amplified barcode molecules. The fluorophore associated with the detection probe can be detected using two-dimensional or three-dimensional fluorescence imaging.

### Barcode Construct and Introduction to Cells

The barcode sequences can be introduced into cells by transfecting the cells with a pooled viral vector library comprising the barcode sequence or the barcode sequence-containing oligonucleotide. For example, a genome of one cell, at least one cell, or each cell of the plurality of cells may comprise the barcode sequence.

In some embodiments, the barcode sequence is integrated into a genome of one, at least one, or each of the plurality of cells. Integrating the barcode sequence can be achieved at a specific site of the genome, e.g., a ROSA26 locus. In some embodiments, integrating the barcode sequence can be achieved by transfecting a cell with a donor plasmid comprising the barcode sequence. Transfecting the cell with the donor plasmid may be achieved by transfecting the cell with the donor plasmid and a plasmid capable of expressing Cas9 and/or a guide ribonucleic acid (gRNA) for integrating the barcode sequence into the genome of one cell, at least one cell, or each of the plurality of cells at a specific site of the cell genome.

In some embodiments, integrating the barcode sequence can be achieved by using a viral vector comprising the barcode sequence. The viral vector may be a retrovirus vector, a lentivirus vector, a gammaretroviral vector, an adenovirus vector, or an adeno-associated virus (AAV) vector. The viral vector can be injected into an organism or a tissue of the organism.

### Fixation

Prior to the barcode sequence detection, the cell, at least one cell, or the plurality of cells comprising the barcode sequences (for example, a biological sample or a tissue sample) may be processed, e.g., fixed. The fixative may comprise a non-cross-linking fixative, such as a precipitating fixative (e.g., an alcohol, such as methanol or ethanol), a denaturing fixative (e.g., a weak acid, such as acetic acid), or a combination thereof. Fixation of the cells or the biological sample may be performed by fixing the plurality of cells without using a cross-linking fixative. The plurality of cells can comprise live cells and/or dead cells prior to fixation.

The fixative can comprise methanol (or another alcohol, or another precipitating fixative) and acetic acid (or another weak acid). The ratio of methanol and acetic acid in fixative can be, for example, from about 10:1 (e.g., v/v, w/w, v/w, or w/v) to about 1:10 (e.g., v/v, w/w, v/w, or w/v). The fixative can comprise, for example, from about 5% acetic acid in methanol to about 75% acetic acid in methanol (e.g., v/v, w/w, v/w, and w/v).

The fixative can comprise a cross-linking fixative, such as formaldehyde (e.g., such as 3%-4% formaldehyde in phosphate-buffered saline) and glutaraldehyde.

The biological sample comprising the plurality of cells that is ready for the barcode detection methods described herein can also be a formalin-fixed, paraffin-embedded (FFPE) sample or specimen. FFPE is a widely used form of preservation and preparation for biopsy specimens that aid in examination, experimental research, and diagnostic/drug development. An FFPE tissue sample is first preserved by fixing the sample in formaldehyde (formalin) to preserve the proteins and vital structures within the tissue. Next, it is embedded in a paraffin wax block, termed an FFPE block. This step makes it easier to cut or microdissect slices of required sizes to mount on a microscopic slide for examination. The barcode sequence detection methods described herein can be suitable for a FFPE sample or specimen.

### Applications

The barcode sequence detection methods described herein can be suitable for various applications. A couple of exemplary, non-limiting examples are described below.

The methods can be used in high throughput screening. Cellular phenotypes can be assayed, for example, by imaging and connected to genetic or environmental perturbations that can be identified by barcode sequences. For this, large numbers of conditions or perturbations can be analyzed in parallel by in situ imaging. Additionally, dynamic phenotypes can be recovered by using time-lapse imaging to analyze the temporal dynamics of cellular behaviors, with end-point analysis of barcodes in the same cell.

The methods also can be used in the analysis of clonal dynamics and heterogeneity in tumors, immunology, organ transplantation, infectious diseases, cell therapies (e.g., CAR-T), and developmental biology. It may help to decipher the lineage structure of tumors and metastases and its relationship to the spatial organization of the tumor. The in situ detection would reveal the spatial organization, thereby allowing in situ analysis of tumor clonal lineage for biomedical research and clinical applications. Similar approaches can be employed in characterizing immune system development and tissue development.

The in situ detection methods described herein can be better understood by reference to the Examples that follow, but those skilled in the art will appreciate that these are only illustrative of the invention as described more fully in the numbered embodiments and claims that follow.

### EXAMPLES

### Example 1: Detection of Barcodes in Tumor Tissues

32-40-mer RNA barcodes were computationally designed as each comprising two independent equally sized fragments according to the following criteria:
having a GC content between 30-70%;
having a melting temperature (Im) of RNA/DNA duplexes greater than 42° C. and less than 65° C.;
having no blastn hits with a melting temperature above 32° C. in both the human and mouse genomes; and
containing no disfavored nucleotide pairs in their ligation junctions.

A second barcode (“imaging barcode”) was designed for the padlock backbone with the following parameters:
28-mer in length;
having a GC content between 30-70%;
containing only bases G/C/A;
having a melting temperature greater than 60° C.; and
having no blastn hits with a melting temperature above 42° C. in both the human and mouse genomes.

RNA barcodes were randomly associated with guide RNAs of interest and were ordered as one-piece oligonucleotide pools from Twist Bioscience (South San Francisco, CA). Oligonucleotide pools were amplified by limited cycle PCR (polymerase chain reaction), cloned in CROPSeq backbones, and packaged in a lentivirus following the protocol as described previously (Feldman et al., 2019).

Cancer cells were infected with the RNA barcode containing lentivirus at a low MOI (<10% infection rates) and either selected with antibiotics or fluorescence-activated cell sorting (FACS) prior to functional experiments (see, e.g., Joung et al., 2017). After 5 days of antibiotic cell selection, cells were dissociated from the cell plates, pelleted, and resuspended in appropriate media and inserted by either subcutaneous injection or orthotopic implantation in immunocompetent or nude mice.

Once the tumor had reached an appropriate size (e.g., when the palpable tumor reached 1 cm for subcutaneous; or 3-4 weeks for other tumor models given a standard protocol), mice were euthanized and tissue was harvested following established institutionally approved protocols. The harvested tissue was immediately frozen in OCT (optimal cutting temperature) compounds and stored at −80° C. for later processing.

Frozen tumors were cryosectioned (e.g., 10-30 μm thickness) and placed on positively charged functionalized coverslips for downstream assays. Coverslips were stored at −80° C. for up to one month after sectioning.

Tissue coverslips were processed by heating them to 37° C. for 2 minutes and were immediately fixed for 10-30 minutes in 4% Formaldehyde/1×PBS. Following fixation, the fixed tissue was rinsed 2× in PBS-Tw (PBS+0.01% Tween-20) and placed in 70% ethanol at room temperature for 30 minutes, or stored in 70% ethanol at −20° C. for up to one month.

After removal from ethanol, the fixed tissue was allowed to dry for up to 10 minutes at room temperature before placing the fixed tissue into custom flow cells for downstream processing. Tissue was hybridized in a buffer containing 2×SSC (sodium chloride-sodium citrate buffer), 20% Formamide, 1% Tween-20, 0.8 U/μL Ribolock, 0.2 mg/mL tRNA at 42° C. overnight with 10 nM of each RNA-barcoding binding padlock probe.

After at least 16 hours of hybridization, the tissue was washed twice for 15 minutes in 20% formamide 2×SSC buffer at 42° C. Following the wash, the tissue was rinsed 3 times with PBS-Tw and immediately placed in the following ligation buffer for at least 2 hours at 37° C. (1× SplintR® Ligase buffer, 1.25 U/μL SplintR® Ligase, 0.8 U/μL Ribolock, 0.2 mg/mL bovine serum albumin, BSA).

Following ligation tissue was washed once with PBS-Tw and placed in an amplification buffer (i.e., 1× Phi29 buffer, 5% glycerol, 0.2 mg/mL BSA, 200 μM amplification primer, 625 μM dNTPs, 0.8 U/μL Ribolock, 40 μM aminoallyl-dUTP, Phi29 polymerase 0.5 U/μL) at 30° C. for 2 hours or overnight at room temperature.

Following amplification, the tissue was washed twice in PBS-Tw, and post-fixed for 5 minutes in 4% formaldehyde/1×PBS-Tw. The tissue was rinsed 3 times with PBS-Tw; the tissue could then be stored at 4° C. for weeks.

To visualize barcode signals, bridging oligonucleotides that bind to the padlock backbone barcode sequences were hybridized in 2×SSC, 20% formamide, and 10% dextran sulfate. Bridging oligonucleotides were washed in a 50% Formamide 2×SSC at 37° C. for 10 minutes.

Following bridging oligonucleotide hybridization, fluorescent readout oligos were further added for hybridization at room temperature on an automated microscope in readout buffer comprised of 10% Ethylene carbonate, 2×SSC, 10% dextran sulfate buffer, with 10 nM of each fluorescent readout oligo for 10 minutes. Samples were rinsed in a bridge readout buffer (10% formamide and 2×SSC) at room temperature for 5 minutes. After rinsing, the samples were placed in a stain of PBS-Tw+DAPI (4′,6-diamidino-2-phenylindole) and imaged using an automated microscope (Nikon Ti2 using a Celesta Laser illuminator and a multiband filterset at 20× magnification) for however many rounds of imaging were required to fully decode barcodes.

Detected barcode sequences were computationally decoded following acquisition by extracting all fluorescent barcodes from the imaging data and aligning them to the pre-existing barcode sequence table generated for each experiment. Proper barcode identification was determined when a cell has greater than 3 (cell>3) identified barcodes in it and the plurality of barcodes all match an on-target barcode in the predefined table.

Alternatively, samples were placed under a fluorescent confocal microscope, imaged with either a 10, 20, or 40× objective, and illuminated using the following laser wavelengths: 408, 477, 545, 638, and 750 nm. These laser wavelengths were chosen to illuminate the relevant fluorescent dyes used in the experiment on the readout oligonucleotides: Atto 488, Alexa 545, Alexa 647, and Alexa 750 along with DAPI. Other fluorescent dyes can be substituted and used. A piezo-actuated stage was used to scan through the Z dimension of the tissue sample enabling full 3D reconstructions of tissue barcodes.

The following table shows the various sequences (some with associated fluorescent dye) used in this Example:

| Name | Sequence | SEQ ID NO : |
|---|---|---|
| Readout 1 | CTTGGGTCTCGT/3AlexF750N/ | 1 |
| Readout 2 | CGTGCCTTGTTT/3ATTO647NN/ | 2 |
| Readout 3 | TTTGCGGGTTCT/3ATTO565N/ | 3 |
| Readout 4 | TCTGGTCCCTCT/3AlexF488N/ | 4 |
| Digit | CACAGAAGCCGAcaACGAGACCCAAG | 5 |
| Adapter 1 | | |
| Digit | AGCAGCAAGAACcaAAACAAGGCACG | 6 |
| Adapter 2 | | |
| Digit | GGAGAACCGAACcaAGAACCCGCAAA | 7 |
| Adapter 3 | | |
| Digit | ACGCGAAACCACcaAGAGGGACCAGA | 8 |
| Adapter 4 | | |
| Digit | AGCGCGAAGAAAcaACGAGACCCAAGcaAAACAAGGCACG | 9 |
| Adapter 5 | | |
| Digit | AAGCACGAGAAGcaACGAGACCCAAGcaAGAACCCGCAAA | 10 |
| Adapter 6 | | |
| Digit | AGGCCACAACCAcaACGAGACCCAAGcaAGAGGGACCAGA | 11 |
| Adapter 7 | | |
| Digit | GCACGCCAACAAcaAAACAAGGCACGcaAGAACCCGCAAA | 12 |
| Adapter 8 | | |
| Digit | CACAGAGCGAGAcaAAACAAGGCACGcaAGAGGGACCAGA | 13 |
| Adapter 9 | | |
| Digit | GAGCAAACGACCcaAGAACCCGCAAAcaAGAGGGACCAGA | 14 |
| Adapter 10 | | |
| Digit | ACGCACGCAAGAcaACGAGACCCAAGcaAAACAAGGCACG | 15 |
| Adapter 11 | CaAGAACCCGCAAA | |
| Digit | AGCGACCGACAAcaACGAGACCCAAGcaAAACAAGGCACG | 16 |
| Adapter 12 | caAGAGGGACCAGA | |
| Digit | AACAGAACCACCcaACGAGACCCAAGcaAGAACCCGCAAA | 17 |
| Adapter 13 | caAGAGGGACCAGA | |
| Digit | AAACAGGCAGACcaAAACAAGGCACGcaAGAACCCGCAAA | 18 |
| Adapter 14 | caAGAGGGACCAGA | |
| Digit | CCAGCAAACAGAcaACGAGACCCAAGcaAAACAAGGCACG | 19 |
| Adapter 15 | caAGAACCCGCAAAcaAGAGGGACCAGA | |
| Digit | CCAAGCAGCGAAcaACGAGACCCAAG | 20 |
| Adapter 16 | | |
| Digit | GAACCGGACACAcaAAACAAGGCACG | 21 |
| Adapter 17 | | |
| Digit | AGGCACCAACACcaAGAACCCGCAAA | 22 |
| Adapter 18 | | |
| Digit | AACGGCAAAGGAcaAGAGGGACCAGA | 23 |
| Adapter 19 | | |
| Digit | AAGCAGGACCGAcaACGAGACCCAAGcaAAACAAGGCACG | 24 |
| Adapter 20 | | |
| Digit | CAGAGAAGGCGAcaACGAGACCCAAGcaAGAACCCGCAAA | 25 |
| Adapter 21 | | |
| Digit | AACAGCAGGAGGcaACGAGACCCAAGcaAGAGGGACCAGA | 26 |
| Adapter 22 | | |
| Digit | AGAAGCAGACGAcaAAACAAGGCACGcaAGAACCCGCAAA | 27 |
| Adapter 23 | | |
| Digit | ACGCACAAGGAAcaAAACAAGGCACGcaAGAGGGACCAGA | 28 |
| Adapter 24 | | |
| Digit | AGGAAGACGGAGcaAGAACCCGCAAAcaAGAGGGACCAGA | 29 |
| Adapter 25 | | |
| Digit | CAAGAACGCGCAcaACGAGACCCAAGcaAAACAAGGCACG | 30 |
| Adapter 26 | caAGAACCCGCAAA | |
| Digit | AAAGCGGAAGGCcaACGAGACCCAAGcaAAACAAGGCACG | 31 |
| Adapter 27 | caAGAGGGACCAGA | |
| Digit | CGAACAGACGGAcaACGAGACCCAAGcaAGAACCCGCAAA | 32 |
| Adapter 28 | caAGAGGGACCAGA | |
| Digit | AAGGAGACGAGCcaAAACAAGGCACGcaAGAACCCGCAAA | 33 |
| Adapter 29 | caAGAGGGACCAGA | |
| Digit | GACAGAACGCACcaACGAGACCCAAGcaAAACAAGGCACG | 34 |
| Adapter 30 | caAGAACCCGCAAAcaAGAGGGACCAGA | |
| Digit | AAAGGACGGCAGcaACGAGACCCAAG | 35 |
| Adapter 31 | | |
| Digit | AGACCGCACACAcaAAACAAGGCACG | 36 |
| Adapter 32 | | |
| Digit | AGACGACAGGAAcaAGAACCCGCAAA | 37 |
| Adapter 33 | | |
| Digit | ACCAGCGGAAAGcaAGAGGGACCAGA | 38 |
| Adapter 34 | | |
| Digit | GGCAGCCACAAAcaACGAGACCCAAGcaAAACAAGGCACG | 39 |
| Adapter 35 | | |
| Digit | GACCAACAAACGcaACGAGACCCAAGcaAGAACCCGCAAA | 40 |
| Adapter 36 | | |
| Digit | CGAACACAGACGcaACGAGACCCAAGcaAGAGGGACCAGA | 41 |
| Adapter 37 | | |
| Digit | CAAGGCAAGCAAcaAAACAAGGCACGcaAGAACCCGCAAA | 42 |
| Adapter 38 | | |
| Digit | ACAAACGGCCAGcaAAACAAGGCACGcaAGAGGGACCAGA | 43 |
| Adapter 39 | | |
| Digit | CGAAGCACACAGcaAGAACCCGCAAAcaAGAGGGACCAGA | 44 |
| Adapter 40 | | |
| Digit | AGACAGAAACGGcaACGAGACCCAAGcaAAACAAGGCACG | 45 |
| Adapter 41 | caAGAACCCGCAAA | |
| Digit | AAGAGGCAGAGCcaACGAGACCCAAGcaAAACAAGGCACG | 46 |
| Adapter 42 | caAGAGGGACCAGA | |
| Digit | ACCACCAGAGCAcaACGAGACCCAAGcaAGAACCCGCAAA | 47 |
| Adapter 43 | caAGAGGGACCAGA | |
| Digit | ACAAGGCGAGACcaAAACAAGGCACGcaAGAACCCGCAAA | 48 |
| Adapter 44 | caAGAGGGACCAGA | |
| Digit | AAGACAACAGCGcaACGAGACCCAAGcaAAACAAGGCACG | 49 |
| Adapter 45 | caAGAACCCGCAAAcaAGAGGGACCAGA | |
| Barcode 1 | /5Phos/tataccqcgccaacacccgttggttggttcttc | 50 |
| | cgggtctctgtGCCGTCCTGCTTggctaatcgctgagga | |
| Barcode 2 | /5Phos/ttgggataagggctgtgttgttcttcgtggtcg | 51 |
| | tgcgtcgtcggGCCGTCCTGCTTacaagtctgccgacca | |
| Barcode 3 | /5Phos/tataggcaggttgggaggccgtggtttcgggtg | 52 |
| | tgttcttctggGCCGTCCTGCTTagaagtacgcgtgcct | |
| Barcode 4 | /5Phos/ttacggacgcaagggattctctcttgttgggct | 53 |
| | tgtgtggctcgGCCGTCCTGCTTaaggcggagagtacga | |
| Barcode 5 | /5Phos/tagcgaagaaagggctttccctcgcttgcgtct | 54 |
| | ctttcgttgctGCCGTCCTGCTTagccctcttgagacca | |
| Barcode 6 | /5Phos/aacccgaaagctccagttgcttgtttctcgggt | 55 |
| | tggtgtgcgctGCCGTCCTGCTTaccgtatggccagaca | |
| Barcode 7 | /5Phos/aatgagcctctgacggggttggcctcttccgtt | 56 |
| | tgtgtggctctGCCGTCCTGCTTacctaaccgcccaaga | |
| Barcode 8 | /5Phos/aagccctgccataaacgttctctgtcgttcggc | 57 |
| | ggttccttgcgGCCGTCCTGCTTcttggccaggagaaca | |
| Barcode 9 | /5Phos/aaggaggtcggaccattcggtttctcttccggt | 58 |
| | tgggttgcgcgGCCGTCCTGCTTttgagcgagaagggct | |
| Barcode 10 | /5Phos/aagacatgggccaagggtccgccgtttggtctt | 59 |
| | ggtgttgtcctGCCGTCCTGCTTagtgtgggctaatgga | |
| Barcode 11 | /5Phos/ttgcgggctgactcatggttgcgccctgtcttt | 60 |
| | cccgtctttctGCCGTCCTGCTTaaccaggaatgggact | |
| Barcode 12 | /5Phos/aagacggtaccgcatgtctgccttgtgttcctt | 61 |
| | gcgttgggcctGCCGTCCTGCTTcccaacaacagagcct | |
| Barcode 13 | /5Phos/taggaccaccaatagctctgctggtgttgtccg | 62 |
| | ctgtttggtccGCCGTCCTGCTTacaggatggcacgtgt | |
| Barcode 14 | /5Phos/taacaagggagtggcatccggttcgttcttgtc | 63 |
| | cttgtcgcctgGCCGTCCTGCTTatgtaactggcgcgct | |
| Barcode 15 | /5Phos/atgcacaatggcgactttcttccttcgtgctgc | 64 |
| | tccgtgttcgcGCCGTCCTGCTTcagggtccaggaagaa | |
| Barcode 16 | /5Phos/tagaaggagtgccaacgggtgctcgcttcggtc | 65 |
| | gtgttcttgtgGCCGTCCTGCTTacgagagaccacccat | |
| Barcode 17 | /5Phos/ttagtgcgcctgggatgctccctctggtttctt | 66 |
| | ggtcgtggcttGCCGTCCTGCTTtcaccgggaatagcca | |
| Barcode 18 | /5Phos/aatacaacggcctgccttgctccttcctgttgt | 67 |
| | gttgcctgtcgGCCGTCCTGCTTctgagacggaactcca | |
| Barcode 19 | /5Phos/ttgaagctcctggccagcgctttggtctccctg | 68 |
| | ggtttcctggtGCCGTCCTGCTTtcaggaacgggtacaa | |
| Barcode 20 | /5Phos/taagaggagccgtaactgttgttctgccgcctc | 69 |
| | ttctcgtcgtgGCCGTCCTGCTTggacacgaagggctta | |
| Barcode 21 | /5Phos/taggacgatgcgacagggctcgtgtttctgcgc | 70 |
| | gtcgtttgtccGCCGTCCTGCTTgccggcagatgaagaa | |
| Barcode 22 | /5Phos/tacgagtgggaagacatgtggcctcgtttggtc | 71 |
| | ttcgcgtttgtGCCGTCCTGCTTtgcaaagccgggcata | |
| Barcode 23 | /5Phos/atagggccgcgagaatttcttgtctgcccgttg | 72 |
| | cgctttctcttGCCGTCCTGCTTaggatctgcaggagta | |
| Barcode 24 | /5Phos/atctacactagccgccgttctcgttcttccttc | 73 |
| | cttqcgcggttGCCGTCCTGCTTtaggcaatgcagcgga | |
| Bridge 1 | CGTTGGTTGGTTCTTCCGGGTCTCTGacCTTGGGTCTCGT | 74 |
| Bridge 2 | TTGTTCTTCGTGGTCGTGCGTCGTCGacCGTGCCTTGTTT | 75 |
| Bridge 3 | GCCGTGGTTTCGGGTGTGTTCTTCTGacTTTGCGGGTTCT | 76 |
| Bridge 4 | TCTCTCTTGTTGGGCTTGTGTGGCTCacTCTGGTCCCTCT | 77 |
| Bridge 5 | TCCCTCGCTTGCGTCTCTTTCGTTGCacCTTGGGTCTCGT | 78 |
| | acCGTGCCTTGTTT | |
| Bridge 6 | TGCTTGTTTCTCGGGTTGGTGTGCGCacCTTGGGTCTCGT | 79 |
| | acTTTGCGGGTTCT | |
| Bridge 7 | GTTGGCCTCTTCCGTTTGTGTGGCTCacCTTGGGTCTCGT | 80 |
| | acTCTGGTCCCTCT | |
| Bridge 8 | TTCTCTGTCGTTCGGCGGTTCCTTGCacCGTGCCTTGTTT | 81 |
| | acTTTGCGGGTTCT | |
| Bridge 9 | CGGTTTCTCTTCCGGTTGGGTTGCGCacCGTGCCTTGTTT | 82 |
| | acTCTGGTCCCTCT | |
| Bridge 10 | TCCGCCGTTTGGTCTTGGTGTTGTCCacTTTGCGGGTTCT | 83 |
| | acTCTGGTCCCTCT | |
| Bridge 11 | GTTGCGCCCTGTCTTTCCCGTCTTTCacCTTGGGTCTCGT | 84 |
| | acCGTGCCTTGTTTacTTTGCGGGTTCT | |
| Bridge 12 | CTGCCTTGTGTTCCTTGCGTTGGGCCacCTTGGGTCTCGT | 85 |
| | acCGTGCCTTGTTTacTCTGGTCCCTCT | |
| Bridge 13 | CTGCTGGTGTTGTCCGCTGTTTGGTCacCTTGGGTCTCGT | 86 |
| | acTTTGCGGGTTCTacTCTGGTCCCTCT | |
| Bridge 14 | CCGGTTCGTTCTTGTCCTTGTCGCCTacCGTGCCTTGTTT | 87 |
| | acTTTGCGGGTTCTacTCTGGTCCCTCT | |
| Bridge 15 | TCTTCCTTCGTGCTGCTCCGTGTTCGacCTTGGGTCTCGT | |
| | acCGTGCCTTGTTTacTTTGCGGGTTCTacTCTGGTCCCT | |
| | CT | |
| Bridge 16 | GGTGCTCGCTTCGGTCGTGTTCTTGTacCTTGGGTCTCGT | 89 |
| Bridge 17 | CTCCCTCTGGTTTCTTGGTCGTGGCTacCGTGCCTTGTTT | 90 |
| Bridge 18 | TGCTCCTTCCTGTTGTGTTGCCTGTCacTTTGCGGGTTCT | 91 |
| Bridge 19 | CGCTTTGGTCTCCCTGGGTTTCCTGGacTCTGGTCCCTCT | 92 |
| Bridge 20 | GTTGTTCTGCCGCCTCTTCTCGTCGTacCTTGGGTCTCGT | 93 |
| | acCGTGCCTTGTTT | |
| Bridge 21 | GCTCGTGTTTCTGCGCGTCGTTTGTCacCTTGGGTCTCGT | 94 |
| | acTTTGCGGGTTCT | |
| Bridge 22 | GTGGCCTCGTTTGGTCTTCGCGTTTGacCTTGGGTCTCGT | 95 |
| | acTCTGGTCCCTCT | |
| Bridge 23 | TCTTGTCTGCCCGTTGCGCTTTCTCTacCGTGCCTTGTTT | 96 |
| | acTTTGCGGGTTCT | |
| Bridge 24 | TTCTCGTTCTTCCTTCCTTGCGCGGTacCGTGCCTTGTTT | 97 |
| | acTCTGGTCCCTCT | |

### Example 2: Tracking Therapeutic Products

The aforementioned barcoding system can be inserted into therapeutic cells and gene therapy products (e.g., CAR-T, NK, iPSC, AAV, and Lentivirus) for measurements of product engraftment, expansion, and persistence. Such information can be used retrospectively for understanding factors leading to a product's successful uptake. Information from these assays can ultimately be used to engineer superior products in the future.

### Example 3: Compound Screening

The aforementioned barcoding system can be used in cell culture or organoid systems for enabling rapid optical and sequencing-based measurements of cell population responses to potential drug products. This information can be used to screen more effective drugs for a given treatment.

### EXEMPLARY EMBODIMENTS

Embodiment 1. A method for detecting barcode sequences in a biological sample in situ, said method comprising the steps of:
a) processing a biological sample comprising a plurality of cells, wherein each cell of the plurality of cells comprises an oligonucleotide having a single barcode sequence of 20 to 60 nucleotides, so that a plurality of barcode molecules, each of which comprises the barcode sequence, are amplified in the plurality of cells of the biological sample in situ, and
b) detecting the plurality of barcode molecules so that the barcode sequences are located in situ.

Embodiment 2. The method of embodiment 1, wherein the biological sample is a mammalian tumor tissue.

Embodiment 3. The method of embodiment 1 or embodiment 2, wherein the oligonucleotide further comprises a CRISPR gRNA.

Embodiment 4. The method of any one of embodiments 1 to 3, wherein the barcode sequence is operably linked to a promoter.

Embodiment 5. The method of embodiment 4, wherein the promoter is a bacteriophage T3 promoter, a bacteriophage T7 promoter, or a bacteriophage SP6 promoter.

Embodiment 6. The method of any one of embodiments 1 to 5, wherein the biological sample is obtained by
i. preparing a pooled viral vector library comprising the oligonucleotides;
ii. transforming the pooled viral vector library to a plurality of cells; and
iii. obtaining a biological sample comprising the plurality of transformed cells.

Embodiment 7. The method of embodiment 6, wherein the pooled viral vector library is delivered to a mammal so that a plurality of mammalian cells are transformed with the pooled viral vector library, and wherein the biological sample is obtained from the mammal.

Embodiment 8. The method of embodiment 6, wherein the pooled viral vector library is transformed into a plurality of cultured mammalian cells, wherein the transformed cultured mammalian cells are transferred to a mammal, and wherein the biological sample is obtained from the mammal.

Embodiment 9. The method of embodiment 6, wherein the pooled viral vector library is constructed from a viral vector wherein the viral vector is a retrovirus vector, a lentivirus vector, a gammaretroviral vector, an adenovirus vector, or an adeno-associated virus (AAV) vector.

Embodiment 10. The method of embodiment 9, wherein the retrovirus vector, the lentivirus vector, or the gammaretroviral vector is a non-integrating viral vector or a self-inactivating retroviral vector.

Embodiment 11. The method of embodiment 6, wherein the viral vector further comprises a cDNA or a CRISPR gRNA.

Embodiment 12. The method of any one of embodiments 1 to 11, wherein the single barcode sequence comprises 20 to 40 nucleotides.

Embodiment 13. The method of embodiment 12, wherein the barcode sequence
i. has a GC content of about 30% to about 70%; and
ii. has a Tm in a range of about 42° C. to about 75° C.

Embodiment 14. The method of embodiment 13, wherein the Tm is about 60° C. or greater.

Embodiment 15. The method of any one of embodiments 1 to 14, wherein detecting the plurality of barcode molecules in situ is achieved by in situ nucleic acid sequencing or fluorescence in situ hybridization.

Embodiment 16. The method of any one of embodiments 1 to 15, wherein step a) further comprises:
i. transcribing the barcode sequence to produce an RNA molecule;
ii. annealing the RNA molecule to a padlock probe, wherein the padlock probe comprises an imaging barcode flanked by two RNA-binding sequences, and wherein each RNA-binding sequence is complementary to a section of the barcode sequence;
iii. ligating the padlock probe to form a single chain circular DNA molecule; and
iv. performing rolling circle replication of the single chain circular DNA molecule to amplify the barcode molecule.

Embodiment 17. The method of embodiment 16, wherein detecting the plurality of barcode molecules is achieved by performing (i) in situ sequencing of the barcode molecule, or (ii) in situ hybridization against the barcode molecule, a branched DNA bound to the barcode molecule, or the imaging barcode.

Embodiment 18. The method of embodiment 17, wherein the in situ hybridization is fluorescence in situ hybridization (FISH).

Embodiment 19. The method of embodiment 16, wherein the padlock probe comprises a spacer oligonucleotide placed between the imaging barcode and at least one of the two RNA-binding sequences.

Embodiment 20. The method of embodiment 16, wherein the imaging barcode has a length of 28 bases, and wherein each of the two RNA-binding sequences has a length of 7-20 bases.

Embodiment 21. The method of embodiment 16, wherein each of two RNA-binding sequences
i. has a GC content of about 30% to about 70%;
ii. contains only guanosine (G), cytosine (C), and adenine (A); and
iii. has a Tm in a range of about 42° C. to about 65° C.

Embodiment 22. The method of embodiment 16, wherein the padlock probe is designed so that an interaction between the padlock probe and the barcode sequence has a Levenshtein distance greater than 4 and has a Tm greater than 42° C.

Embodiment 23. The method of embodiment 2, further comprising a step of fixing the mammalian tumor tissue before the step a).

Embodiment 24. The method of embodiment 23, wherein the fixed mammalian tumor tissue is a formalin-fixed paraffin-embedded (FFPE) tissue.

What is claimed is:

1. A method for detecting barcode sequences in a biological sample in situ, said method comprising the steps of:
c) processing a biological sample comprising a plurality of cells, wherein each cell of the plurality of cells comprises an oligonucleotide having a single barcode sequence of 20 to 60 nucleotides, so that a plurality of barcode molecules, each of which comprises the barcode sequence, are amplified in the plurality of cells of the biological sample in situ, and
d) detecting the plurality of barcode molecules so that the barcode sequences are located in situ.

2. The method of claim 1, wherein the biological sample is a mammalian tumor tissue.

3. The method of claim 1, wherein the oligonucleotide further comprises a CRISPR gRNA.

4. The method of claim 1, wherein the barcode sequence is operably linked to a promoter.

5. The method of claim 4, wherein the promoter is a bacteriophage T3 promoter, a bacteriophage T7 promoter, or a bacteriophage SP6 promoter.

6. The method of claim 1, wherein the biological sample is obtained by
iv. preparing a pooled viral vector library comprising the oligonucleotides;
v. transforming the pooled viral vector library to a plurality of cells; and
vi. obtaining a biological sample comprising the plurality of transformed cells.

7. The method of claim 6, wherein the pooled viral vector library is delivered to a mammal so that a plurality of mammalian cells are transformed with the pooled viral vector library, and wherein the biological sample is obtained from the mammal.

8. The method of claim 6, wherein the pooled viral vector library is transformed into a plurality of cultured mammalian cells, wherein the transformed cultured mammalian cells are transferred to a mammal, and wherein the biological sample is obtained from the mammal.

9. The method of claim 6, wherein the pooled viral vector library is constructed from a viral vector wherein the viral vector is a retrovirus vector, a lentivirus vector, a gammaretroviral vector, an adenovirus vector, or an adeno-associated virus (AAV) vector.

10. The method of claim 9, wherein the retrovirus vector, the lentivirus vector, or the gammaretroviral vector is a non-integrating viral vector or a self-inactivating retroviral vector.

11. The method of claim 6, wherein the viral vector further comprises a cDNA or a CRISPR gRNA.

12. The method of claim 1, wherein the single barcode sequence comprises 20 to 40 nucleotides.

13. The method of claim 12, wherein the barcode sequence
iii. has a GC content of about 30% to about 70%; and
iv. has a Tm in a range of about 42° C. to about 75° C.

14. The method of claim 13, wherein the Tm is about 60° C. or greater.

15. The method of claim 1, wherein detecting the plurality of barcode molecules in situ is achieved by in situ nucleic acid sequencing or fluorescence in situ hybridization.

16. The method of claim 1, wherein step a) further comprises:
v. transcribing the barcode sequence to produce an RNA molecule;
vi. annealing the RNA molecule to a padlock probe, wherein the padlock probe comprises an imaging barcode flanked by two RNA-binding sequences, and wherein each RNA-binding sequence is complementary to a section of the barcode sequence;
vii. ligating the padlock probe to form a single chain circular DNA molecule; and
viii. performing rolling circle replication of the single chain circular DNA molecule to amplify the barcode molecule.

17. The method of claim 16, wherein detecting the plurality of barcode molecules is achieved by performing (i) in situ sequencing of the barcode molecule, or (ii) in situ hybridization against the barcode molecule, a branched DNA bound to the barcode molecule, or the imaging barcode.

18. The method of claim 17, wherein the in situ hybridization is fluorescence in situ hybridization (FISH).

19. The method of claim 16, wherein the padlock probe comprises a spacer oligonucleotide placed between the imaging barcode and at least one of the two RNA-binding sequences.

20. The method of claim 16, wherein the imaging barcode has a length of 28 bases, and wherein each of the two RNA-binding sequences has a length of 7-20 bases.

21. The method of claim 16, wherein each of two RNA-binding sequences
iv. has a GC content of about 30% to about 70%;
v. contains only guanosine (G), cytosine (C), and adenine (A); and
vi. has a Tm in a range of about 42° C. to about 65° C.

22. The method of claim 16, wherein the padlock probe is designed so that an interaction between the padlock probe and the barcode sequence has a Levenshtein distance greater than 4 and has a Tm greater than 42° C.

23. The method of claim 2, further comprising a step of fixing the mammalian tumor tissue before the step a).

24. The method of claim 23, wherein the fixed mammalian tumor tissue is a formalin-fixed paraffin-embedded (FFPE) tissue.
